(19) United States
(12) Patent Application Publication
(10) Pub. No.: US 2025/0249155 A1
(43) Pub. Date: Aug. 7, 2025

(54) BREAST PUMP

(71) Applicant: SHENZHEN ZXD TRADING CO., LTD, Shenzhen (CN)

(72) Inventor: Wei Yi, Shenzhen (CN)

(21) Appl. No.: 18/648,399

(22) Filed: Apr. 28, 2024

(30) Foreign Application Priority Data
Feb. 5, 2024 (CN) 202410173306.0

(51) Int. Cl. A61M 1/06

(52) U.S. Cl. CPC A61M 1/067; A61M 2205/36; A61M 2205/8237

(57) ABSTRACT

A breast pump is disclosed. The breast pump includes a body and a heating assembly. The body defines a channel. The heating assembly includes an attaching member and a charging device. The attaching member is electrically connected to the charging device. The attaching member is at least partially arranged in the channel. The charging device is connected to the body. The heating assembly is connected to the body. The attaching member is located on a side of the body that is capable of contacting with a breast and configured to contact with the breast. The heating assembly is further configured to generate heat through the charging device.

### CROSS REFERENCE TO RELATED APPLICATIONS

This application claims priority to Chinese Patent Application No. 202410173306.0, filed on Feb. 5, 2024, the contents of which are herein incorporated by reference in their entirety.

### TECHNICAL FIELD

The present disclosure relates to the technical field of milk articles, and in particular to a breast pump.

### BACKGROUND

Breasts are highly sensitive parts of a human body. Breast pumps work by creating negative pressures in the interiors, such that milk is able to be sucked out and further stored. When the ambient temperature is low, if the breasts directly contact with the breast pumps, it is easy for a user to have a strong feeling of coldness, resulting in physical discomfort and also leading to a decrease in milk production.

### SUMMARY

In one aspect, a breast pump is provided. The breast pump includes a body, defining a channel; and a heating assembly, connected to the body and including: an attaching member, at least partially arranged in the channel, located on a side of the body, and configured to contact with the breast; and a charging device, connected to the body and electrically connected to the attaching member. The attaching member is further configured to generate heat through the charging device.

In another aspect, a breast pump is provided. The breast pump includes a body, defining a channel configured to receive a breast and allow a nipple to pass therethrough; and a heating assembly, connected to the body and including: an attaching member, attached to cover the channel, and configured to contact with the breast; and a charging device, detachably connected to the body and electrically connected to the attaching member. The charging device is configured to supply power to the attaching member to enable the attaching member to generate heat.

In a further aspect, a breast pump is provided. The breast pump includes a body, having an attaching surface and defining a channel configured to receive a breast and allow a nipple to pass therethrough; and a breast housing, attached to the attaching surface, and including an inserting portion inserted into the channel and attached to the channel. The breast housing is configured to contact with the breast and capable of generating heat.

### BRIEF DESCRIPTION OF THE DRAWINGS

FIG. 1 is a schematic assembly view of a breast pump according to some embodiments of the present disclosure.

FIG. 2 is a schematic exploded view of the breast pump shown in FIG. 1 according to some embodiments of the present disclosure.

FIG. 3 is a schematic exploded view of the breast pump shown in FIG. 1 viewed from another perspective according to some embodiments of the present disclosure.

FIG. 4 is a schematic exploded view of an attaching member provided by some embodiments of the present disclosure.

FIG. 5 is a schematic structural view of a heating element provided by some embodiments of the present disclosure.

FIG. 6 is a schematic view illustrating the routing of a heating wire inside the heating element provided by some embodiments of the present disclosure.

FIG. 7 is another schematic exploded view of the breast pump shown in FIG. 1 according to some embodiments of the present disclosure.

FIG. 8 is a schematic enlarged view of an area A circumscribed in FIG. 7.

FIG. 9 is a schematic planar assembly view of a breast pump provided by some embodiments of the present disclosure.

FIG. 10 is a schematic enlarged view of an area B circumscribed in FIG. 9.

100, heating assembly; 11, attaching member; 111, attaching flange; 112, engaging rib; 113, first attaching element; 114, second attaching element; 115, heating element; 1151, terminal; 1152, electric heating portion; 01, unit piece; 011, outer ring; 012, inner ring; 02, gap; 116, protrusion; 12, charging device; 13, first bending portion; 14, second bending portion; 200, body; 21, channel; 22, attaching surface; 300, fixing member; 31, first magnetic attraction element; 32. a second magnetic attraction element; 33, supporting leg or bracket; 34, transverse portion; 400, button.

### DETAILED DESCRIPTION

In order to enable those skilled in the art to better understand the technical solution of the present disclosure, the present disclosure is described in detail below in conjunction with the accompanying drawings. The description in this part is only exemplary and explanatory, and should not be regarded as having any restrictive effect on the scope of protection of the present disclosure.

It should be noted that similar reference numerals and letters indicate similar items in the drawings below. Thus, once an item is defined in one drawing, it does not need to be further defined and explained in subsequent drawings.

It should be noted that, orientations or positional relationships indicated by the terms “center”, “up”, “down”, “left”, “right”, “vertical”, “horizontal”, “inner”, “outer”, etc., are based on the orientations or positional relationships shown in the drawing, or are the orientations or positional relationships in which the product of the present disclosure are customarily placed when in use, and are only for the convenience of describing the present disclosure and simplifying the description, and do not indicate or imply that the device or element referred to must have specific orientations or be constructed and operated in particular orientations, and therefore, it cannot be construed as a limitation on the present disclosure. In addition, the terms “first”, “second”, “third”, etc., are used only used for differentiation and should not be construed as indicating or implying relative importance.

In addition, the terms such as “horizontal”, “vertical”, “overhanging”, etc., do not imply that the components are required to be absolutely horizontal or overhanging, but instead the components may be slightly titled. For example, “horizontal” simply means that its orientation is more horizontal than that of “vertical”; it does not mean that the structure must be completely horizontal, but may be slightly tilted.

Unless otherwise defined, all technical and scientific terms used herein have the same meanings as commonly understood by those skilled in the art of the present disclosure. The terms used in the specification of the present disclosure herein are only used for the purpose of describing specific embodiments and are not intended to limit the present disclosure.

The present disclosure is described in further detail below in conjunction with the accompanying drawings.

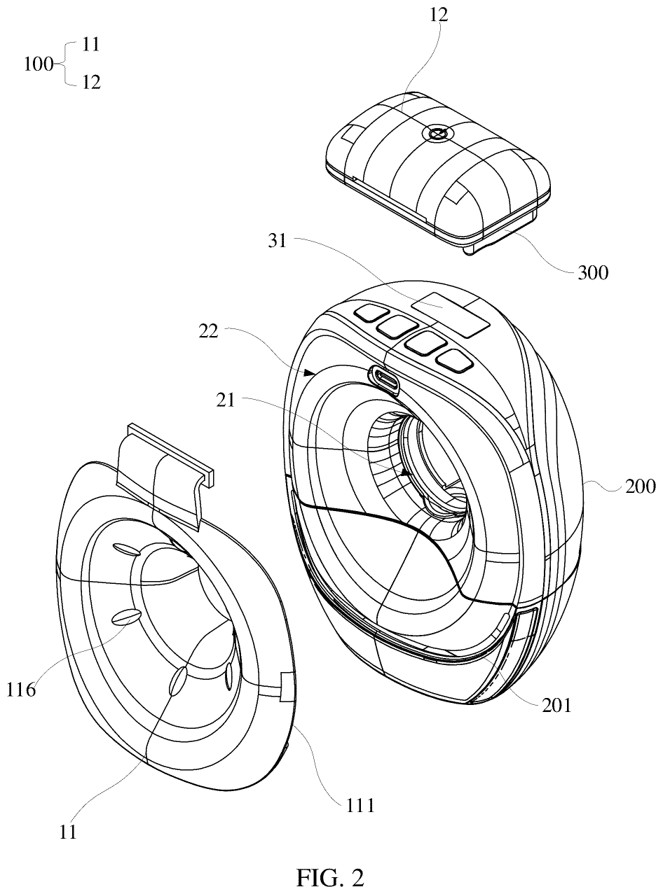

FIG. 2 shows a breast pump according to some embodiments of the present disclosure. As shown in FIG. 2, a breast pump may include a body 200 and a heating assembly 100. A channel 21 may be defined in the body 200. The channel 21 may be configured to accommodate or receive a breast and allow a nipple to pass therethrough. The heating assembly 100 may generate heat by itself, and then be attached to or contact with the breast. The heating assembly 100 may include an attaching member 11 and a charging device 12. The attaching member 11 may be electrically connected to the charging device 12. The attaching member 11 may be at least partially arranged or received in the channel 21. The charging device 12 may be connected to the body 200. In some embodiments, the heating assembly 100 is connected to the body 200. The attaching member 11 is located on a side of the body 200 that is configured to contact with or capable of contacting with the breast, and configured to contact with the breast. The attaching member 11 may generate heat through the charging device 12. The heating assembly 100 is configured to be attached to the breast, thereby reducing discomfort caused by the breast being directly attached to the body 200. The attaching member 11 is a member of the heating assembly 100 that is configured to directly contact with the breast. The attaching member 11 is attached to cover the surface of the channel 21. A rechargeable battery is arranged inside the charging device 12. The rechargeable battery may provide energy required for heat generation of the attaching member 11. The charging device 12 may be attached or adhered to the body 200 via glue or an adhesive tape. The charging device 12 may also be engaged with the body 200 via engagement structures. The charging device 12 may also attract the body 200 by a suction cup, a magnet, or other means.

In the breast pump according to some embodiments of the present disclosure, the heating assembly 100 is disposed on the body 200. When in use, the heating assembly 100 heats up a part of the breast pump that contacts with the breast via electric heating, so as to improve the comfort of a user in the process of sucking or pumping milk. The heating assembly 100 further includes the attaching member 11 and the charging device 12. The attaching member 11 is made of a soft and elastic material such as silicone, rubber, and the like. In this way, the user may feel more comfortable when the attaching member 11 is in contact with the breast. The rechargeable battery is arranged inside the charging device 12 to supply energy or power for the heat generation of the attaching member 11. When the charging device 12 is in a battery-low state, the charging device 12 may be charged. In this way, the attaching member 11 is enabled to generate heat continuously in a relatively cold environment. Thus, it is possible to reduce the feeling of discomfort due to the stimulation to the breast caused by the feeling of coldness when the breast is in contact with the attaching member 11, which in turn reduces the problem of the decrease in milk production due to the discomfort of the breast.

Technical details of each component are described below.

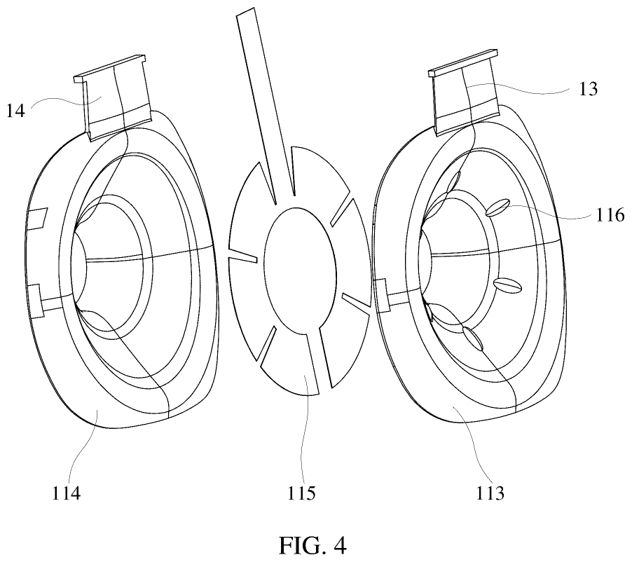

In some embodiments, as shown in FIG. 2 and FIG. 4, the attaching member 11 includes a first attaching element 113, a heating element 115, and a second attaching element 114. The first attaching element 113 is configured to contact with the breast. The second attaching element 114 is configured to contact with the body 200 and is at least partially located in the channel 21. The heating element 115 is electrically connected to the charging device 12. The heating element 115 is disposed or sandwiched between the first attaching element 113 and the second attaching element 114. In some embodiments, the first attaching element 113 and the second attaching element 114 have sheet-like structures and may be made of skin-friendly soft materials such as rubber, silicone, or the like. The heating element 115 is an electric heating piece, which may continuously convert electric energy into thermal energy, such that the temperature of each of the first attaching element 113 and the second attaching element 114 that are attached to two opposite sides of the heating element 115 increases. The heating element 115 is sandwiched between the first attaching element 113 and the second attaching element 114, such that the temperature of the entire attaching member 11 may be uniformly increased. Besides, the heating element 115 is generally made of metal, and it is possible to reduce pollution or erosion to the heating element 115 caused by external liquids, since the heating element 115 is sandwiched between the first attaching element 113 and the second attaching element 114.

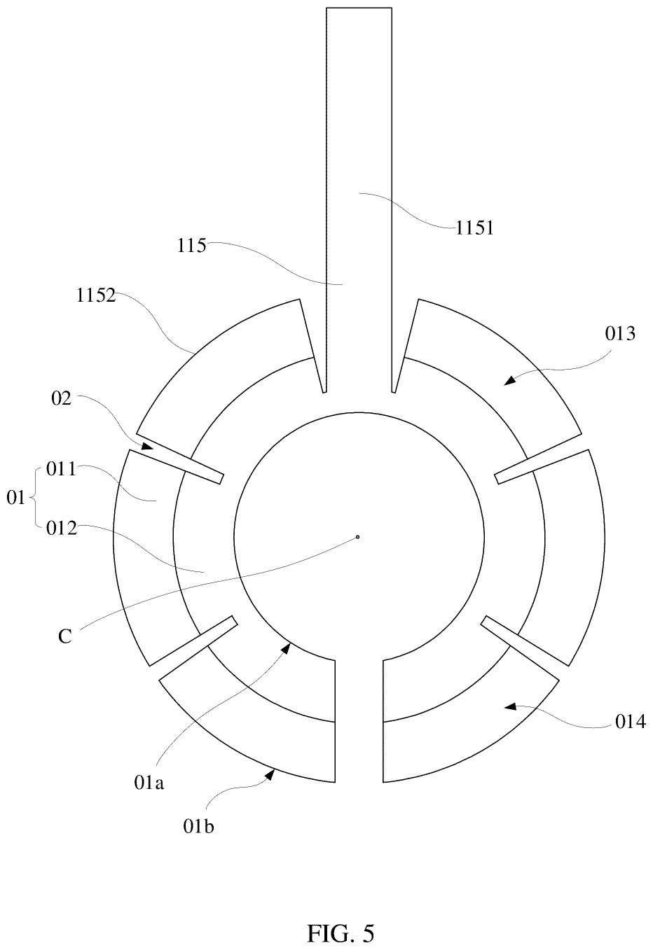

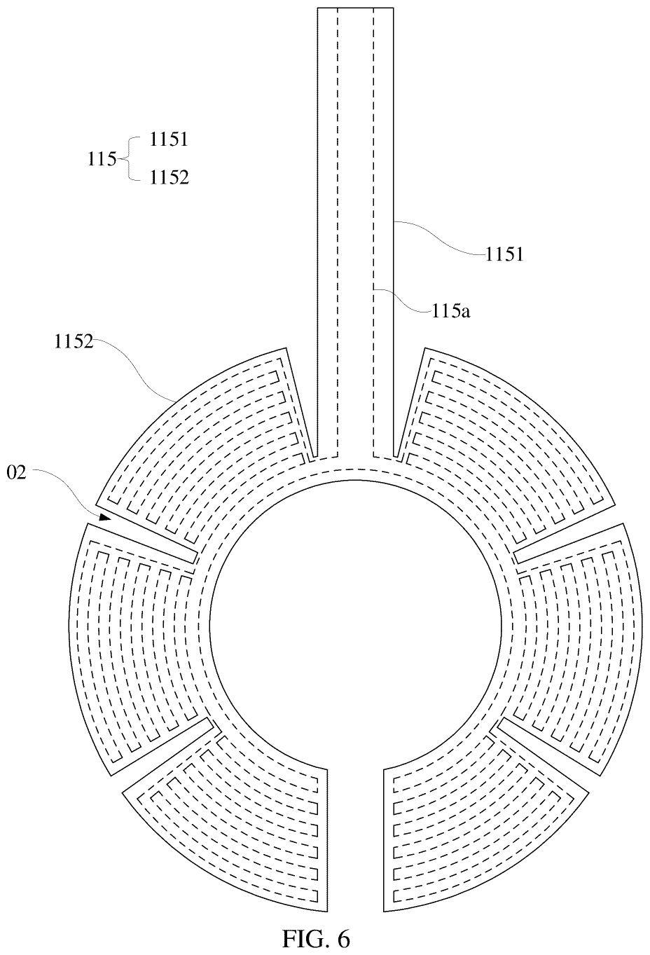

As shown in FIG. 4 and FIG. 5, the heating element 115 includes an electric heating portion 1152 and a terminal 1151. The electric heating portion 1152 is electrically connected to the terminal 1151. The terminal 1151 is electrically connected to the charging device 12. The electric heating portion 1152 is configured to generate heat, and the terminal 1151 is configured to be connected to the charging device 12. In some embodiments, the electric heating portion 1152 includes a plurality of unit pieces 01 (also called “heating sub-portion 01”) that are distributed around a surrounding center C or in a circular manner and connected in series with each other. A gap 02 is defined between every adjacent two of the plurality of unit pieces 01. In some embodiments, the plurality of unit pieces 01 are disposed or distributed around the surrounding center C along a circular trajectory, and the center of the circular trajectory is the surrounding center C. One end of each of the plurality of unit pieces 01 which is close to the surrounding center C is a series end 01a, another end of each of the plurality of unit pieces 01 away from the surrounding center C is a free end 01b. Along a direction from the series end 01a to the free end 01b, the width of each of the plurality of unit pieces 01 gradually increases. The every adjacent two of the plurality of unit pieces 01 are electrically connected to each other in series through the series ends 01a, and all the unit pieces 01 are configured to generate heat after being connected in series. As shown in FIG. 6, broken lines shown in FIG. 6 illustrate the routing of a heating wire 115a inside the heating element 115. As shown in FIG. 6, the heating wire 115a is evenly detoured inside each of the plurality of unit pieces 01, such that each of the plurality of unit pieces 01 may evenly generate heat. Two ends of the heating wire 115a pass through the inner side of the terminal 1151 and are electrically connected to the charging device 12.

In practical application, the electric heating portion 1152 may be bent when the electric heating portion 1152 is sandwiched or clamped between the first attaching element 113 and the second attaching element 114. The free ends 01b of adjacent unit pieces 01 may get close to or approach each other during bending; at this time, the gap 02 defined between adjacent unit pieces 01 may provide an avoidance space for the free ends 01b of the adjacent unit pieces 01. With this design, it is possible to reduce the damage of the electric heating portion 1152 caused by a wrinkle of the electric heating portion 1152 generated in the bending process due to the overtight configuration. The heating wire 115a inside the heating element 115 is evenly detoured inside each of the plurality of unit pieces 01, such that each of the plurality of unit pieces 01 may evenly generate heat. In this way, the heating element 115 may transmit heat stably and evenly to the first attaching element 113 and the second attaching element 114.

In some embodiments, as shown in FIG. 5, each of the plurality of unit pieces 01 includes an inner ring 012 and an outer ring 011. The inner ring 012 is disposed at a side of each of the plurality of unit pieces 01 close to the surrounding center C. The outer ring 011 is disposed at a side of each of the plurality of unit pieces 01 that is away from the surrounding center C. Both the outer ring 011 and the inner ring 012 are configured to generate heat, but the outer ring 011 and the inner ring 012 are electrically connected to the charging device 12 via series circuits independent of each other. That is to say, the outer rings 011 of all the plurality of unit pieces 01 are connected in series to form a first series circuit, the inner rings 012 of all the plurality of unit pieces 01 are connected in series to form a second series circuit, and the first series circuit are independent from the second series circuit. In this way, the inner ring 012 and the outer ring 011 may be controlled independently, such that the inner ring 012 and the outer ring 011 may generate different intensities of heat when in use, which is convenient for the user to adjust the temperatures of the inner ring 012 and the outer ring 011 separately or independently according to his/her own feelings.

In some embodiments, the aforementioned plurality of unit pieces 01 distributed around or in a circular manner includes at least two fast-heating elements 013 (that is, at least two of the plurality of unit pieces 01 are implemented as fast-heating elements 013, which is also called “first heating sub-element 013”), a detouring density or distribution density of the heating wire 115a inside each of the at least two fast-heating elements 013 is greater than that of the other unit pieces 01 (also called “second heating sub-element 014”). The at least two fast-heating elements 013 are symmetrically distributed about the terminal 1151. The at least two fast-heating elements 013 may generate heat faster than the other unit pieces 01 (the second heating sub-element 014). When in use, the at least two fast-heating elements 013 are located on a side of the breast near or close to the head of the user. In practical application, since the side of the breast near the head of the user is farther away from the body than other portions of the breast, it is more difficult for the side of the breast near the head of the user to receive heat from the body than other portions of the breast. By using the at least two fast-heating elements 013, the electric heating portion 1152 may accumulate heat more quickly on the side of the breast near the head of the user to make up for the insufficiency of heat supplied by the body. In this way, various portions of the breast may be at a relatively balanced temperature during the milk-sucking or milk-pumping process, which improves the comfort of the breast during the milk-sucking process, thereby helping the breast to produce more milk.

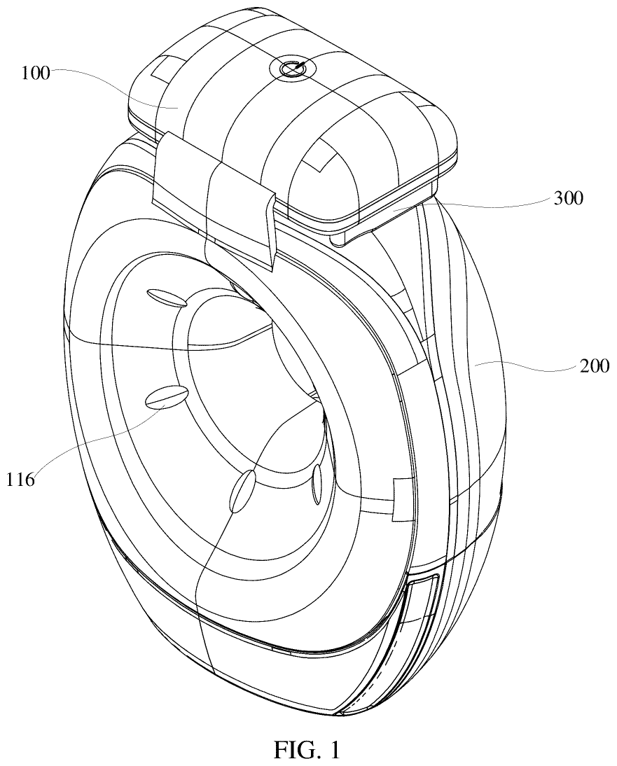

In some embodiments, as shown in FIG. 1 and FIG. 4, a plurality of protrusions 116 are arranged on one side of the attaching member 11 configured to contact with the breast. The plurality of protrusions 116 on the attaching member 11 are arranged around or surround the channel 21. In some embodiments, the plurality of protrusions 116 are smooth protrusions arranged on the side of the attaching member 11 configured to contact with the breast, and configured to massage and stimulate the breast during the milk-sucking process.

During the milk-sucking process, the plurality of protrusions 116 may have the function of massaging the breast. Under the effect of massage, the discharge of the milk of the breast may be facilitated, which in turn facilitates the breast pump to suck out the milk.

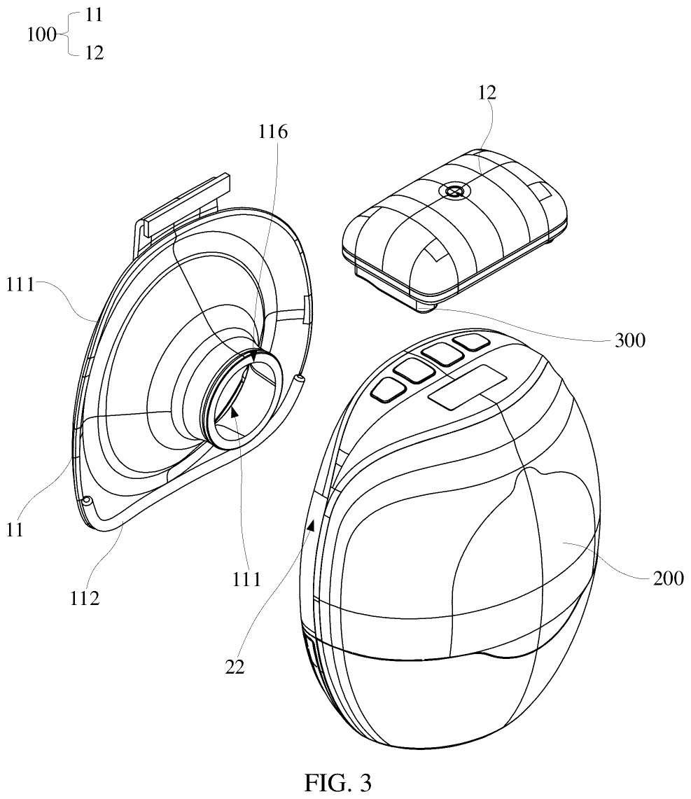

In some embodiments, as shown in FIG. 3, the attaching member 11 includes an engaging rib 112. The engaging rib 112 may be arranged on one side of the attaching member 11 close to the body 200. In some embodiments, the engaging rib 112 is made of silicone or rubber, and may be snapped or engaged into the gap or an engaging groove via the deformation of the engaging rib 112.

The body 200 includes an engaging groove 201, and the engaging groove 201 may be correspondingly engaged with the engaging rib 112. In some embodiments, the engaging groove 201 may be a notch or groove defined on the body 200. The engaging rib 112 may be engaged or snapped into the engaging groove 201 via compressional deformation of the engaging rib 112.

The engaging rib 112 is disposed around an edge of the side of the attaching member 11 close to the body 200. Since the attaching member 11 itself is relatively soft, the edge is not easy to fix. When the user is holding the breast pump, the edge of the attaching member 11 is easy to be detached from the body 200. By arranging the engaging rib 112 on the edge of the attaching member 11 and defining the engaging groove 201 on the body 200, in the assembly processing, the edge of the attaching member 11 may be engaged into the engaging groove 201 through the engaging rib 112 to fix the edge, and thus it is possible to effectively prevent the edge of the attaching member 11 from being detached from the body 200, and the stability of the connection between various components of the breast pump may be further enhanced.

In some embodiments, as shown in FIG. 2 and FIG. 3, the body 200 includes an attaching surface 22 surrounding the channel 21. The attaching member 11 includes an attaching flange 111 surrounding the channel 21. The attaching flange 111 may be tightly attached to the attaching surface 22. In some embodiments, the attaching flange 111 is an edge structure of the attaching member 11. The attaching surface 22 is an arc surface of the body 200 attached to the attaching flange 111. After the attaching surface 22 is attached to the attaching flange 111, a sealed structure may be formed between the edge of the attaching member 11 and the body 200. In this way, pollution to the milk and the breast pump itself caused by pollutants, such as dust and liquid, which enter the space defined between the attaching member 11 and the body 200, may be reduced, and the health and hygiene of the breast pump in use are guaranteed.

In some embodiments, as shown in FIG. 3 and FIG. 4, the charging device 12 is positioned or located at the top of the body 200. The heating assembly 100 further includes a first bending portion 13 and a second bending portion 14 in a bending shape. The first attaching element 113 is connected to the charging device 12 via the first bending portion 13, and the second attaching element 114 is connected to the charging device 12 via the second bending portion 14. The heating element 115 is further sandwiched between the first bending portion 13 and the second bending portion 14. In some embodiments, the first bending portion 13 and the second bending portion 14 may be made of soft elastic materials such as silicone, rubber, or the like. The first bending portion 13 may be configured to fix the first attaching element 113 to the charging device 12, and the second bending portion 14 may be configured to fix the second attaching element 114 to the charging device 12. An electrical connection structure configured to electrically connect the heating element 115 to the charging device 12 are clamped or sandwiched between the first bending portion 13 and the second bending portion 14. As shown in FIG. 4, the heating element 115 may extend to form the electrical connection structure (e.g., the terminal 151), and the electrical connection structure is further electrically connected to the charging device 12. Of course, it is also possible to connect the heating element 115 to the charging device 12 through a wire, a circuit board, or the like. The first bending portion 13 and the second bending portion 14 may clamp the electrical connection structure configured to electrically connect the heating element 115 to the charging device 12 therebetween, which may provide a good protection to the electrical connection structure. Besides, the bending degree of each part of the electrical connection structure may be more uniform in the bending process, and it is possible to reduce the risk of breakage of the electrical connection structure caused by an excessive bending angle at parts of the electrical connection structure.

In some embodiments, as shown in FIG. 1, the breast pump further includes a fixing member 300. The fixing member 300 is arranged between the charging device 12 and the body 200 to connect the charging device 12 and the body 200. The battery is arranged inside the charging device 12, which makes the charging device 12 itself has a certain weight. The fixing member 300 may enable the charging device 12 to be more firmly connected to the body 200. Thus, when in use, the charging device 12 is not easy to fall off from the body 200.

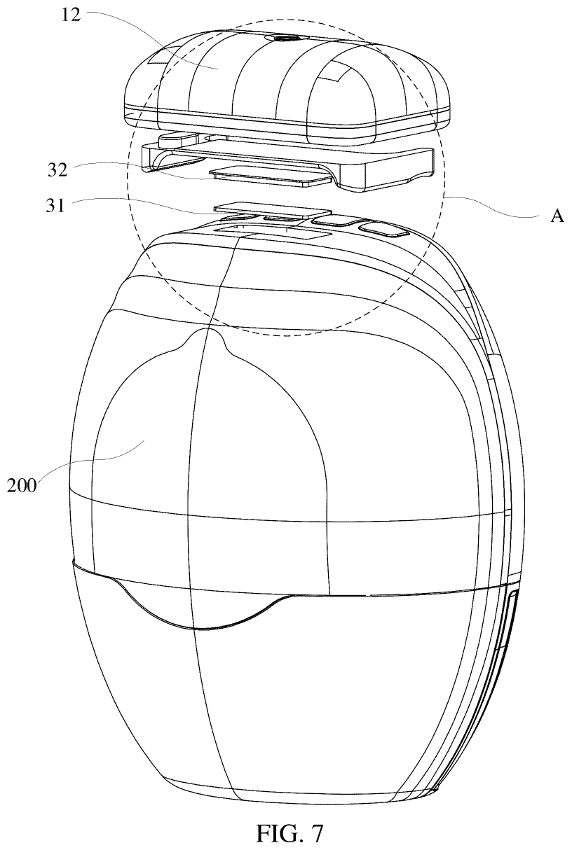

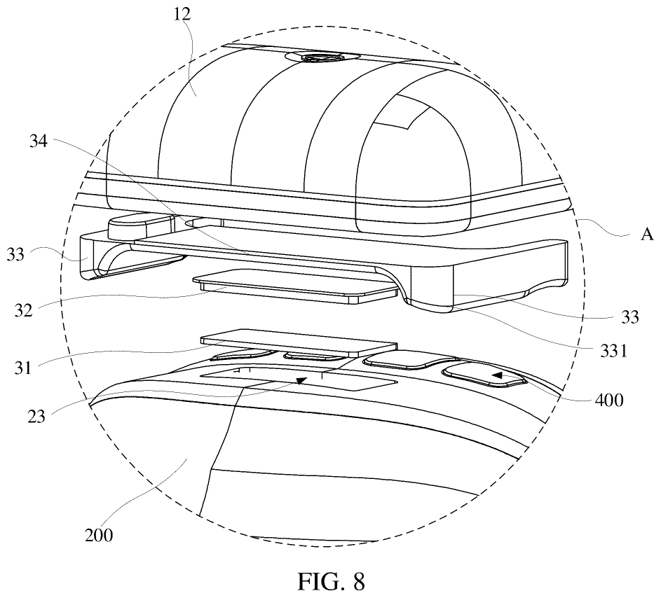

In some embodiments, as shown in FIG. 7 and FIG. 8, the fixing member 300 includes a first magnetic attraction element 31 and a second magnetic attraction element 32. The first magnetic attraction element 31 is arranged on one of the body 200 and the charging device 12, and the second magnetic attraction element 32 is arranged on the other of the body 200 and the charging device 12. The first magnetic attraction element 31 and the second magnetic attraction element 32 are able to attract each other. In some embodiments, the first magnetic attraction element 31 and the second magnetic attraction element 32 may be implemented as a magnet and a magnetic component, respectively. The magnetic component refers to the component that is attractable by the magnet made of metals such as iron, nickel, or the like. In case that the first magnetic attraction element 31 is a magnet, the second magnetic attraction element is a magnetic component. In case that the first magnetic attraction element 31 is a magnetic component, the second magnetic attraction element 32 is a magnet. Of course, it is also possible that both the first magnetic attraction element 31 and the second magnetic attraction element 32 are magnets.

As shown in FIG. 7 and FIG. 8, the first magnetic attraction element 31 is arranged on the body 200, and the second magnetic attraction element 32 is arranged on the charging device 12. Of course, in some embodiments, the first magnetic attraction element 31 may be arranged on the charging device 12, and the second magnetic attraction element 32 may be arranged on the body 200. The charging device 12 is fixed on the body 200 by magnetic attraction, which facilitates the assembly and disassembly of the charging device 12. The fixing structure is simple, and it is not easy to be damaged.

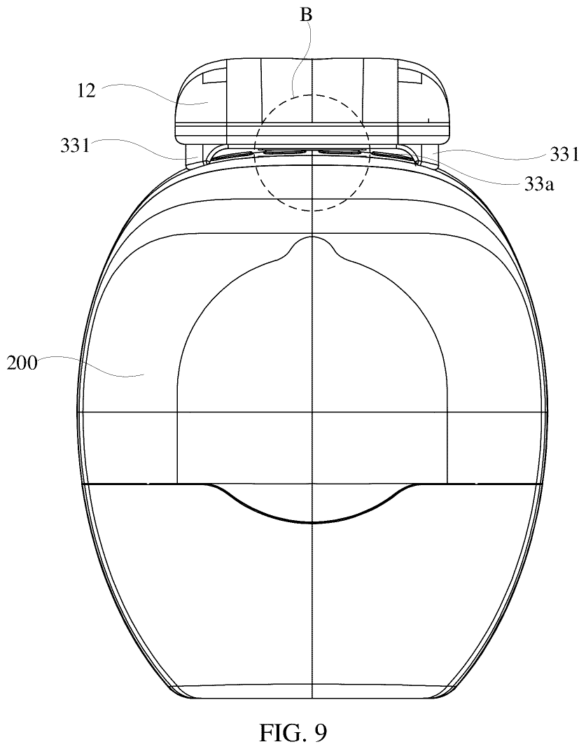

In some embodiments, as shown in FIGS. 8-9, the fixing member 300 further includes a supporting leg 33 (also called “bracket”, “supporting element”, etc.). One end of the supporting leg 33 is connected to the charging device 12, and the other end of the supporting leg 33 abuts against the body 200, such that an avoidance space 33a is defined between the charging device 12 and the body 200. In some embodiments, the avoidance space 33a is a gap existing between the charging device 12 and the body 200.

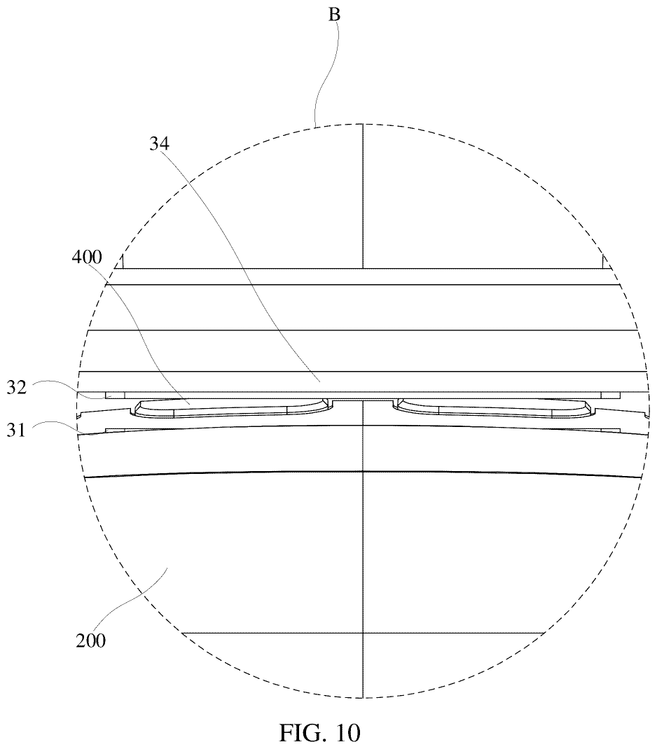

As shown in FIG. 10, the breast pump further includes a button 400. The button 400 is arranged on the body 200 and is located in the avoidance space 33a. This design may reduce the interference to the breast pump caused by the charging device 12 squeezing or pressing the button 400 during the assembly. The supporting leg 33 may support the charging device 12 at the top, such that a certain gap exists between the bottom of the charging device 12 and the button 400, thereby reducing the possibility that the charging device 12 squeezing or pressing the button 400.

In some embodiments, as shown in FIG. 8 and FIG. 9, the fixing member 300 further includes a transverse portion 34 (also called “connection portion”). Two ends of the transverse portion 34 are connected to the support leg 33. The middle part of the transverse portion 34 is attached to the charging device 12. In some embodiments, the transverse portion 34 may be made of elastic materials such as silicone, rubber, or the like. The transverse portion 34 may be in shape of a strip. Two ends of the transverse portion 34 are fixed on the support leg 33. The first magnetic attraction element 31 may be arranged on one of the transverse portion 34 and the body 200, and the second magnetic attraction element 32 may be arranged on the other of the transverse portion 34 and the body 200. The first magnetic attraction element 31 may attract the second magnetic attraction element 32. In some embodiments, when the first magnetic attraction element 31 and the second magnetic attraction element 32 attract each other, the transverse portion 34 is in an elastically bending state. In some embodiments, the supporting leg 33 may include a pair of sub legs 331 spaced apart from each other. Two ends of the transverse portion 34 are connected to the pair of sub legs 331 in one-to-one correspondence.

As shown in FIG. 5, FIG. 6, FIG. 7, FIG. 8, the first magnetic attraction element 31 is arranged on the body 200, and the second magnetic attraction element 32 is arranged on a side of the transverse portion 34 close to the body 200. Of course, in some embodiments, the first magnetic attraction element 31 may be arranged on the transverse portion 34, and the second magnetic attraction element 32 may be arranged on the body 200. When the charging device 12 is not assembled to the body 200, the transverse portion 34 is attached to a surface of the charging device 12. After the charging device 12 is assembled to the body 200, the surface of the charging device 12 having the transverse portion 34 faces the body 200, and the second magnetic attraction element 32 moves towards the first magnetic attraction element 31 under the attraction of the first magnetic attraction element 31 and further attract and be attached to the first magnetic attraction element 31. In this case, the middle part of the transverse portion 34 is stretched. Since the two ends of the transverse portion 34 are connected to the supporting leg 33 and the supporting leg 33 is further fixed to the charging device 12, the charging device 12 is tightened by the transverse portion 34 and attached to the body 200, such that the charging device 12 is not easy to fall off from the body 200 when in use.

In one aspect, a breast pump is provided. The breast pump includes a body, defining a channel; and a heating assembly, connected to the body and including: an attaching member, at least partially arranged in the channel, located on a side of the body, and configured to contact with the breast; and a charging device, connected to the body and electrically connected to the attaching member. The attaching member is further configured to generate heat through the charging device.

In some embodiments, the attaching member includes: a first attaching element, configured to contact with the breast; a second attaching element, configured to contact with the body and at least partially located in the channel; and a heating element, electrically connected to the charging device, and sandwiched between the first attaching element and the second attaching element.

In some embodiments, the heating element includes: a terminal, electrically connected to the charging device; and an electric heating portion, electrically connected to the terminal and configured to generate heat.

In some embodiments, the electric heating portion includes a plurality of unit pieces that are distributed around a surrounding center and connected in series with each other, and a gap is defined between every adjacent two of the plurality of unit pieces.

In some embodiments, one end of each of the plurality of unit pieces that is close to the surrounding center is a series end, and another end of each of the plurality of unit pieces that is away from the surrounding center is a free end; along a direction from the series end to the free end, the width of each of the plurality of unit pieces gradually increases.

In some embodiments, each of the plurality of unit pieces includes an inner ring and an outer ring, the inner ring is disposed at a side of each of the plurality of unit pieces that is close to the surrounding center, the outer ring is disposed at a side of each of the plurality of unit pieces that is away from the surrounding center, and the outer ring and the inner ring are electrically connected to the charging device via series circuits independent of each other.

In some embodiments, the heating element includes a heating wire detoured inside each of the plurality of unit pieces, the plurality of unit pieces are divided into first heating sub-elements and second heating sub-elements, and a distribution density of parts of the heating wire inside the first heating sub-elements is greater than that of the second heating sub-elements.

In some embodiments, a plurality of protrusions are arranged on a side of the attaching member configured to contact with the breast, and the plurality of protrusions arranged on the attaching member surround the channel.

In some embodiments, the attaching member includes an engaging rib, and the engaging rib is arranged on a side of the attaching member that is close to the body. The body includes an engaging groove, and the engaging groove is engaged with the engaging rib.

In some embodiments, the body includes an attaching surface surrounding the channel, the attaching member includes an attaching flange surrounding the channel, and the attaching flange is attached to the attaching surface.

In some embodiments, the charging device is positioned at the top of the body, the heating assembly further includes a first bending portion and a second bending portion in a bending shape, the first attaching element is connected to the charging device through the first bending portion, the second attaching element connected to the charging device through the second bending portion, and the heating element is sandwiched between the first bending portion and the second bending portion.

In some embodiments, the breast pump further includes a fixing member, and the fixing member is arranged between the charging device and the body and configured to connect the charging device and the body.

In some embodiments, the fixing member includes: a first magnetic attraction element, arranged on one of the body and the charging device; and a second magnetic attraction element, arranged on the other of the body and the charging device. The first magnetic attraction element and the second magnetic attraction element are able to attract each other.

In some embodiments, the fixing member further includes a bracket, one end of the bracket is connected to the charging device, and the other end of the bracket abuts against the body, such that an avoidance space is defined between the charging device and the body. The breast pump further includes a button, and the button is arranged on the body and located in the avoidance space.

In some embodiments, the fixing member further includes a transverse portion, two ends of the transverse portion are both connected to the bracket, and the middle part of the transverse portion is attached to the charging device. The first magnetic attraction element is arranged on one of the transverse portion and the body, the second magnetic attraction element is arranged on the other of the transverse portion and the body, and the first magnetic attraction element and the second magnetic attraction element are able to attract each other. In a case where the first magnetic attraction element and the second magnetic attraction element attract each other, the transverse portion is in an elastically bending state.

In another aspect, a breast pump is provided. The breast pump includes a body, defining a channel configured to receive a breast and allow a nipple to pass therethrough; and a heating assembly, connected to the body and including: an attaching member, attached to cover the channel, and configured to contact with the breast; and a charging device, detachably connected to the body and electrically connected to the attaching member. The charging device is configured to supply power to the attaching member to enable the attaching member to generate heat.

In a further aspect, a breast pump is provided. The breast pump includes a body 200, having an attaching surface 22 and defining a channel 21 configured to receive a breast and allow a nipple to pass therethrough; and a breast housing 11, attached to the attaching surface 21, and including an inserting portion 116 inserted into the channel 21 and attached to the channel 21. The breast housing 116 is configured to contact with the breast and capable of generating heat.

By arranging the heating assembly on the body of the breast pump and the heating assembly including the attaching member configured to be attached to the breast and the charging device configured to store energy required for heat generation, when in use, the attaching member is configured to isolate the breast from the body of the breast pump. Besides, the breast is a sensitive private part, the attaching member may generate heat by itself via the charging device and then contact with the breast in a cold environment, which may reduce the discomfort caused by stimulation to the breast at low temperature. Besides, the amount of milk produced by the breast during one suction process may be increased.

In the description of the present disclosure, it should also be noted that, unless otherwise expressly specified or qualified, terms “arranged”, “mounted”, “connected”, and “coupled” shall be understood in a broad sense, for example, may include a fixed connection, a detachable connection, or an integral connection; may include a mechanical connection or an electrical connections; or may also include a direct connection or an indirect connection via an intervening structure; or may also be an inner communication of two elements. For those skilled in the art, the specific meaning of the above terms in the present disclosure may be understood according to specific situations.

It should be noted that, in the description of the present disclosure, relational terms, such as first, second, and the like, are used only to distinguish one entity or operation from another, and do not necessarily require or imply any such actual relationship or order between those entities or operations. Furthermore, terms “including”, “comprising”, or any other variant thereof, are intended to cover non-exclusive inclusion. Thus, a process, method, article, or apparatus comprising a set of elements includes not only the listed elements, but also other elements not expressly listed, or other elements inherent to the process, method, article, or apparatus. Without further limitation, the fact that an element is defined by the phrase “includes a . . . ” does not preclude the existence of additional identical elements in the process, method, article or apparatus that includes said element. In the absence of further restrictions, the elements defined by the statement “including a . . . ” do not exclude the existence of additional identical elements in the process, method, article, or apparatus that includes the elements.

Although embodiments of the present disclosure have been illustrated and described, for those skilled in the art, it can be understood that, a variety of changes, modifications, substitutions, and variations may be made to these embodiments without departing from the principle and spirit of the present disclosure. The scope of the present disclosure is limited by the appended claims and their equivalents.

What is claimed is:

1. A breast pump, comprising:
a body, defining a channel; and
a heating assembly, connected to the body and comprising:
an attaching member, at least partially arranged in the channel, located on a side of the body, and configured to contact with the breast; and
a charging device, connected to the body and electrically connected to the attaching member; and
wherein the attaching member is further configured to generate heat through the charging device.

2. The breast pump as claimed in claim 1, wherein the attaching member comprises:
a first attaching element, configured to contact with the breast;
a second attaching element, configured to contact with the body and at least partially located in the channel; and
a heating element, electrically connected to the charging device, and sandwiched between the first attaching element and the second attaching element.

3. The breast pump as claimed in claim 2, wherein the heating element comprises:
a terminal, electrically connected to the charging device; and
an electric heating portion, electrically connected to the terminal and configured to generate heat.

4. The breast pump as claimed in claim 3, wherein the electric heating portion comprises a plurality of unit pieces that are distributed around a surrounding center and connected in series with each other, and a gap is defined between every adjacent two of the plurality of unit pieces.

5. The breast pump as claimed in claim 4, wherein one end of each of the plurality of unit pieces that is close to the surrounding center is a series end, and another end of each of the plurality of unit pieces that is away from the surrounding center is a free end;
along a direction from the series end to the free end, the width of each of the plurality of unit pieces gradually increases.

6. The breast pump as claimed in claim 4, wherein each of the plurality of unit pieces comprises an inner ring and an outer ring, the inner ring is disposed at a side of each of the plurality of unit pieces that is close to the surrounding center, the outer ring is disposed at a side of each of the plurality of unit pieces that is away from the surrounding center, and the outer ring and the inner ring are electrically connected to the charging device via series circuits independent of each other.

7. The breast pump as claimed in claim 4, wherein the heating element comprises a heating wire detoured inside each of the plurality of unit pieces, the plurality of unit pieces are divided into first heating sub-elements and second heating sub-elements, and a distribution density of parts of the heating wire inside the first heating sub-elements is greater than that of the second heating sub-elements.

8. The breast pump as claimed in claim 1, wherein a plurality of protrusions are arranged on a side of the attaching member configured to contact with the breast, and the plurality of protrusions arranged on the attaching member surround the channel.

9. The breast pump as claimed in claim 1, wherein the attaching member comprises an engaging rib, and the engaging rib is arranged on a side of the attaching member that is close to the body; and
wherein the body comprises an engaging groove, and the engaging groove is engaged with the engaging rib.

10. The breast pump as claimed in claim 1, wherein the body comprises an attaching surface surrounding the channel, the attaching member comprises an attaching flange surrounding the channel, and the attaching flange is attached to the attaching surface.

11. The breast pump as claimed in claim 2, wherein the charging device is positioned at the top of the body, the heating assembly further comprises a first bending portion and a second bending portion in a bending shape, the first attaching element is connected to the charging device through the first bending portion, the second attaching element connected to the charging device through the second bending portion, and the heating element is sandwiched between the first bending portion and the second bending portion.

12. The breast pump as claimed in claim 1, wherein the breast pump further comprises a fixing member, and the fixing member is arranged between the charging device and the body and configured to connect the charging device and the body.

13. The breast pump as claimed in claim 12, wherein the fixing member comprises:
a first magnetic attraction element, arranged on one of the body and the charging device; and
a second magnetic attraction element, arranged on the other of the body and the charging device; and
wherein the first magnetic attraction element and the second magnetic attraction element are able to attract each other.

14. The breast pump as claimed in claim 13, wherein the fixing member further comprises a bracket, one end of the bracket is connected to the charging device, and the other end of the bracket abuts against the body, such that an avoidance space is defined between the charging device and the body; and
wherein the breast pump further comprises a button, and the button is arranged on the body and located in the avoidance space.

15. The breast pump as claimed in claim 14, wherein the fixing member further comprises a transverse portion, two ends of the transverse portion are both connected to the bracket, and the middle part of the transverse portion is attached to the charging device;
the first magnetic attraction element is arranged on one of the transverse portion and the body, the second magnetic attraction element is arranged on the other of the transverse portion and the body, and the first magnetic attraction element and the second magnetic attraction element are able to attract each other; and
wherein in a case where the first magnetic attraction element and the second magnetic attraction element attract each other, the transverse portion is in an elastically bending state.

16. A breast pump, comprising:
a body, defining a channel configured to receive a breast and allow a nipple to pass therethrough; and
a heating assembly, connected to the body and comprising:
an attaching member, attached to cover the channel, and configured to contact with the breast; and
a charging device, detachably connected to the body and electrically connected to the attaching member; and
wherein the charging device is configured to supply power to the attaching member to enable the attaching member to generate heat.

17. The breast pump as claimed in claim 16, wherein the attaching member comprises:
a first attaching element, configured to contact with the breast;
a second attaching element, configured to contact with the body and at least partially located in the channel; and
a heating element, electrically connected to the charging device, and sandwiched between the first attaching element and the second attaching element.

18. The breast pump as claimed in claim 17, wherein the heating element comprises:
a terminal, electrically connected to the charging device; and
an electric heating portion, electrically connected to the terminal, configured to generate heat, and comprising a plurality of unit pieces that are distributed around a surrounding center and connected in series with each other, wherein a gap is defined between every adjacent two of the plurality of unit pieces.

19. The breast pump as claimed in claim 16, wherein the attaching member comprises an engaging rib, and the engaging rib is arranged on a side of the attaching member that is close to the body; and
wherein the body comprises an engaging groove, and the engaging groove is engaged with the engaging rib.

20. A breast pump, comprising:
a body, having an attaching surface and defining a channel configured to receive a breast and allow a nipple to pass therethrough; and
a breast housing, attached to the attaching surface, and comprising an inserting portion inserted into the channel and attached to the channel, wherein the breast housing is configured to contact with the breast and capable of generating heat.
